(19) United States
(12) Patent Application Publication
(10) Pub. No.: US 2020/0188308 A1
(43) Pub. Date: Jun. 18, 2020

(54) SYSTEMS AND METHODS FOR AUTOMATED PELLET PRESSING AND VIALING

(71) Applicant: RIGHT VALUE DRUG STORES, LLC, Irving, TX (US)

(72) Inventors: Donald Prentiss, Irving, TX (US); Richard E. Appling, II, Irving, TX (US)

(21) Appl. No.: 16/712,351

(22) Filed: Dec. 12, 2019

Related U.S. Application Data
(63) Continuation-in-part of application No. 16/666,720, filed on Oct. 29, 2019.
(60) Provisional application No. 62/823,243, filed on Mar. 25, 2019.
(60) Provisional application No. 62/778,514, filed on Dec. 12, 2018.

(51) Int. Cl. A61K 9/20; B30B 11/04; A61K 9/00

(52) U.S. Cl. CPC A61K 9/2095; B30B 15/302; A61K 9/0024; B30B 11/04

(57) ABSTRACT

Various embodiments of a system for automated pellet pressing and vialing are disclosed herein.

### CROSS REFERENCE TO RELATED APPLICATIONS

This application is a non-provisional application that is a continuation-in-part of U.S. Non-Provisional application Ser. No. 16/666,720 filed on Oct. 29, 2019, it also claims the benefit of U.S. provisional application Ser. No. 62/823,243 filed on Mar. 25, 2019, and U.S. provisional application Ser. No. 62/778,514 filed on Dec. 12, 2018, which are all herein incorporated by reference in their entirety.

### FIELD

The present disclosure generally relates systems and methods for manufacturing vialed pellets; and in particular to systems and methods for automatically pressing and vialing of implantable pellets.

### BACKGROUND

The manufacturing of implantable pellets, such as pellets containing testosterone, require high manufacturing standards to ensure compliance with requirements related to proper pellet shape, pellet surface area, pellet volume, and pellet integrity. In the past, manual pellet presses have been used to manufacture pellets, which can be time consuming and potentially introduce variance in pellet shape, surface, area, volume and integrity during manufacturing. As such, automated methods for manufacturing pellets that meet the stringent standards of manufacturing such implantable pellets are desirable.

These implantable pellets are oftentimes stored and distributed in vials. A vial is generally understood as a plastic or glass vessel or bottle, which may be tube-shaped or cylindrical and used to store or protect a substance such as a medicine, perfume, chemical, and the like. A vial may also be referred to as a phial, container, bottle, or tube. Vials may include single-dose or multi-dose substances or medications. In some cases, vials are enclosed using a cap, stopper, cork, or other such closure mechanism. In addition, the process of manufacturing and vialing a plurality of pellets for individual storage, packaging and distribution may be better automated into a more time and cost effective process.

It is with these observations in mind, among others, that various aspects of the present disclosure were conceived and developed.

### BRIEF DESCRIPTION OF THE DRAWINGS

FIG. 1 is a simplified block diagram of a system for automated pellet fabrication and vialing.

FIG. 2 is a simplified block diagram of a process flow associated with pellet fabrication and vialing.

FIG. 3 is a perspective view of an automated pellet press used in the system for the automated pellet fabrication and vialing of FIG. 1.

FIG. 4 is an exploded view of the automated pellet press of FIG. 3.

FIG. 5 is a front view of the automated pellet press of FIG. 3.

FIG. 6 shows various illustrations of upper and lower punches having different sizes and related dies that may be used with the automated pellet press of FIG. 3.

FIGS. 7A and 7B show before-and-after assembly of upper and lower punches using a simplified front view of the automated pellet press of FIG. 3.

FIGS. 8A and 8B illustrate before-and-after assembly of a face plate and vacuum tubing using a simplified enlarged front view of the automated pellet press of FIG. 3.

FIGS. 9A and 9B illustrate before-and-after assembly of a feed cup and shaker lever in relation to a die using a simplified view of the automated pellet press of FIG. 3.

FIG. 10 illustrates an assembly of the feed cup and shaker lever shown in FIG. 9B in relation to an upper punch of the automated pellet press of FIG. 3.

FIG. 11A illustrates the feed cup and shaker lever situated in alignment when the die is in a “dispensing” position with the die being shown in phantom;

FIG. 11B illustrates the feed cup and shaker lever with an elongated edge of the feed cup being out of alignment when the die is in a “non-dispensing” position;

FIG. 11C illustrates the feed cup and shaker lever with an elongated edge of the feed cup being returned to a “dispensing” position and moving across the die, an operation which facilitates the “eject” function of the automated pellet press of FIG. 3.

FIG. 12 shows an illustration of the lifting cam of the automated pellet press of FIG. 3 defining an eccentric pathway formed along its face.

FIG. 13 is an image of one embodiment of the system of FIG. 1 for automated pellet vialing.

FIG. 14 is a simplified block diagram depicting an exemplary process including a decision tree associated with the pellet vialing framework of FIG. 1.

FIG. 15 is a simplified block diagram showing an example of a computing system that may implement various services, systems, and methods discussed herein.

Corresponding reference characters indicate corresponding elements among the view of the drawings. The headings used in the figures do not limit the scope of the claims.

### DETAILED DESCRIPTION

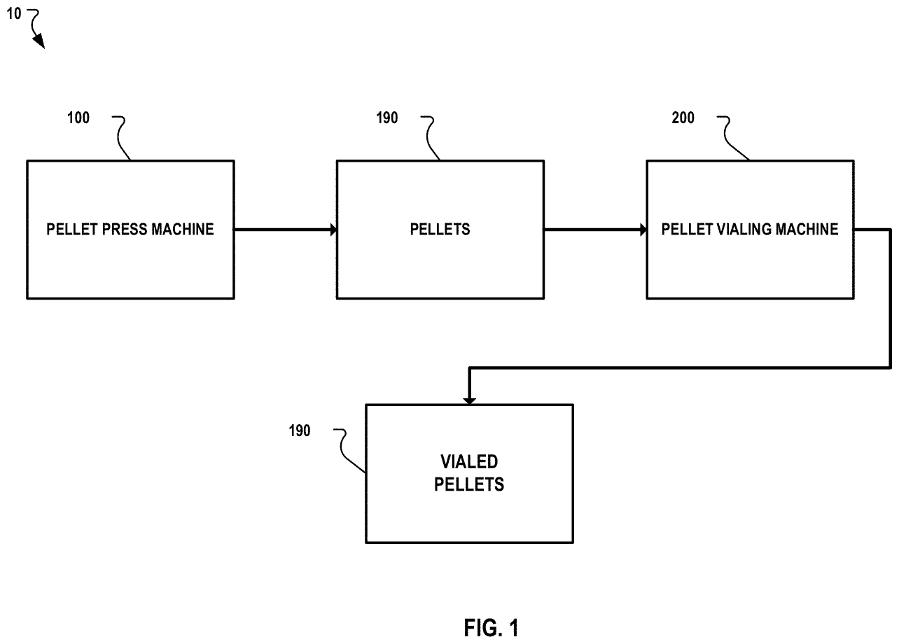

The present disclosure relates to an automated pellet fabrication and vialing system and related method of manufacturing and vialing of implantable pellets. In particular, referring to the drawings, one embodiment of the present system of manufacturing pellets includes an automated pellet press for the automation of pharmaceutical pellet production and an automated pellet vialing and packaging apparatus for the automation of transitioning raw pellets to a vialed and labeled product, is illustrated and generally indicated as 100 in FIGS. 1-15.

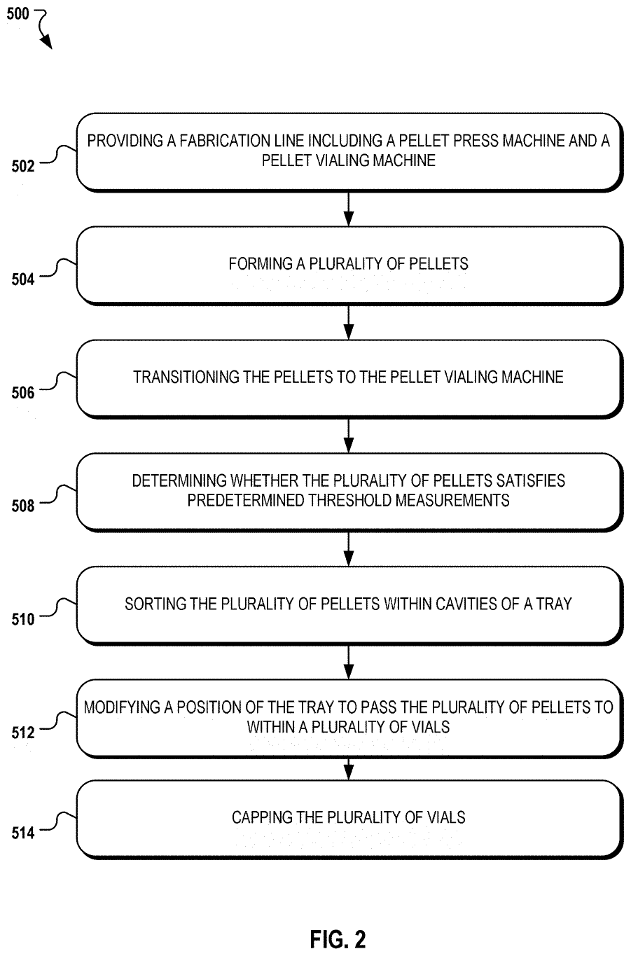

Referring to FIGS. 1 and 2, an exemplary system for automated pellet fabrication, vialing and packaging (hereinafter “system”) 10 is shown. The system 10 may include an automated pellet press 100 which is operable to fabricate a plurality of pellets 190, among other features as described herein. The system 10 may further include a pellet vialing apparatus 200 operable for vialing the plurality of pellets 190 into a plurality of vialed pellets 106 from the plurality of pellets 190 fabricated by the automated pellet press 102. A more detailed description of the system 100 is set for the below.

### Automated Pellet Press

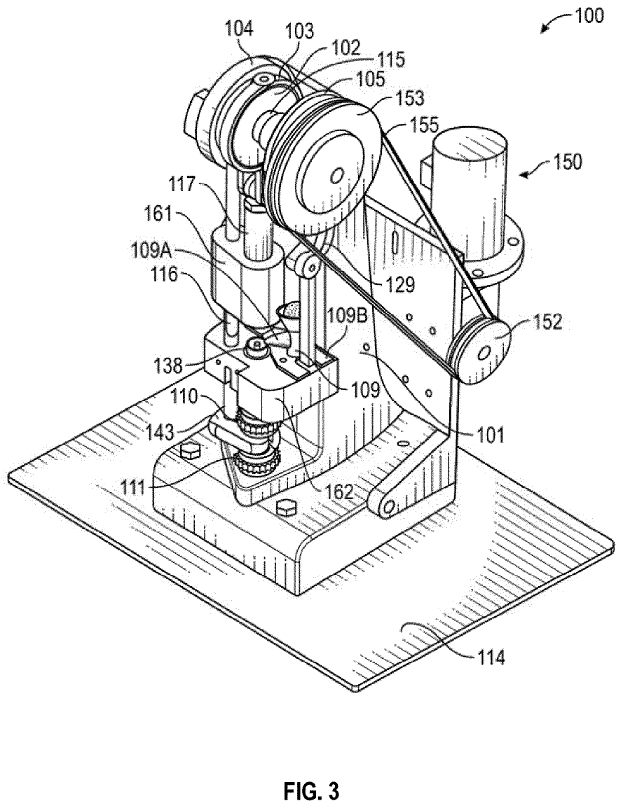

Various embodiments of an automated pellet press are disclosed herein. In some embodiments, the automated pellet press includes a frame operatively connected to a motor having a pulley arrangement that actuates an upper plunger and a lower plunger in alternating opposite axial directions such that an upper punch and a lower punch associated with the upper and lower plungers, respectively, alternately engage a die containing a pharmaceutical compound in powder form to produce an implantable pellet. In some embodiments, the pellets produced by the automated pellet press have the same size, configuration, volume, and pellet integrity to be inserted subcutaneously within a patient for delayed release or release of the pharmaceutical substance over time. Referring to the drawings, an embodiment of the automated pellet press is illustrated and generally indicated as 100 in FIGS. 3-12.

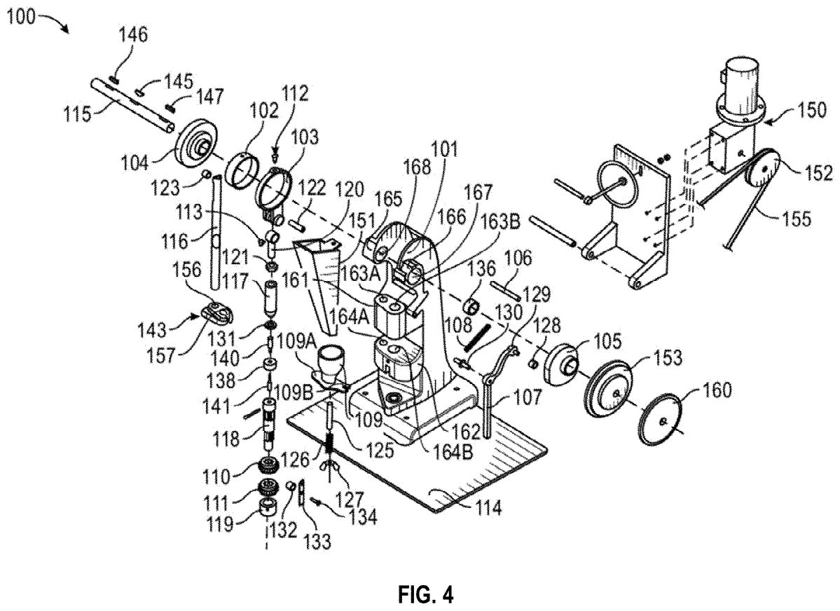

Referring to FIGS. 3 and 4, in some embodiments the automated pellet press 100 is operable to manufacture a plurality of pellets made from a pharmaceutical substance in an automated pressing operation. In one method of manufacture using the automated pellet press 100 a pharmaceutical substance in powder form is poured into a die 138 and then compressed into pellet form when stamped by an upper punch 140 disposed partially within an upper plunger 117 and a lower punch 141 partially disposed within a lower plunger 118 in which the upper and lower punches 140 and 141 are driven against the die 138 in alternating sequence from opposite axial directions. Once stamped, the formed pellet is then extracted from the die 138 for collection.

Referring to FIG. 4, the automated pellet press 100 includes a frame 101 that provides a structure for assembling the components of the automated pellet press 100. In some embodiments, the frame 101 defines an upper mounting portion 161 forming a pair of axially extending channels 163A and 163B as well as a lower mounting portion 162 forming a pair of axially extending channels 164A and 164B in respective alignment with the axially extending channels 163A and 163B. As shown, first and second shoulders 165 and 166 are defined above the upper mounting portion 161 and form aligned respective first and second longitudinal channels 167 and 168 configured to receive a rotatable main shaft 115. In some embodiments, the frame 101 may be secured or rest on a base plate 114 and/or reside within an enclosure (not shown) that prevents contaminants from contacting the pellet during manufacture.

As shown in FIGS. 3-4, a motor 150 is operably coupled to a first pulley 152 which drives a belt 155 engaged between the first pulley 152 and a second pulley 153. The second pulley 153 is coupled to a rotatable main shaft 115 mounted along the first shoulder 165 and a second shoulder 166 of frame 101. In some embodiments, a hand wheel 160 is coupled to one of the end portions of the main shaft 115 and is operable to manually operate the automated pellet press 100 by manually rotating the rotatable main shaft 115 when manual operation is desired.

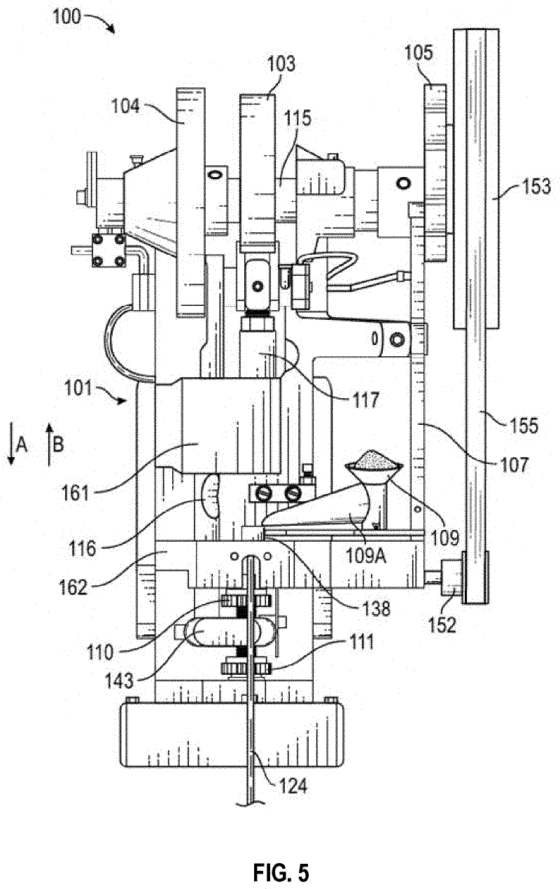

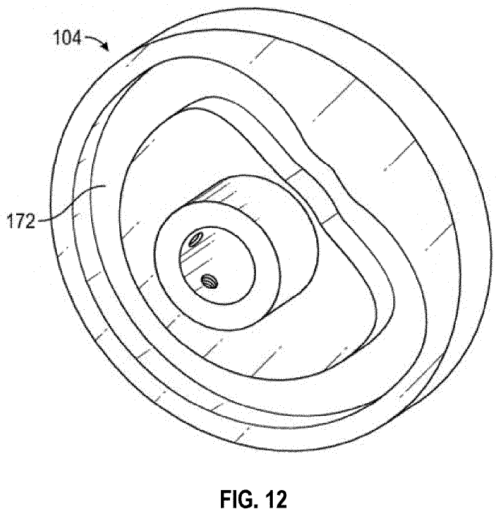

In some embodiments, the rotatable main shaft 115 is coupled to a lifting rod 116 by a converter mechanism which converts a rotational motion provided by the main shaft 115 to an up-and-down reciprocating linear motion of the lifting rod 116. One such embodiment of the converter mechanism is a lifting cam 104 defining an eccentric pathway 172 (shown in FIG. 12) which is operatively coupled to the lifting rod 116 through a laterally extending protrusion 123 defined by or coupled to the lifting rod 116 that is engaged within and follows the eccentric pathway 172 as the rotatable main shaft 115 is rotated. As the laterally extending protrusion 123 follows the path of the eccentric pathway 172 as the main shaft 115 is rotated, the lifting rod 116 is caused to move up and down in opposite axial directions A and B shown in FIG. 5. In some embodiments, a lifting block 143 is attached to the bottom portion of the lifting rod 116 through an aperture 156 formed through the lifting block 143. The lifting block 143 further defines a slot portion 157 configured to engage the lower plunger 118 such that axial movement of the lifting rod 116 causes the lower plunger 118 to concurrently move in the same axial direction. In one embodiment, the lifting rod 116 is disposed through the aligned upper channel 163A and lower channel 164A of frame 101.

As further shown, the main shaft 115 is coupled to the upper plunger 117 by a second converter mechanism which converts the rotational motion provided by the main shaft 115 to a repetitive up and down linear motion of the upper plunger 117 in opposite axial directions A and B. The second converter mechanism may be embodied as an eccentric sheave 102 coupled to the main shaft 115, wherein the eccentric sheave 102 is coaxially engaged within an eccentric strap 103 coupled to an upper plunger eyebolt 120 through an eyebolt pin 122. The upper plunger eyebolt 120 is also coupled to the upper plunger 117 using an eyebolt nut 121. In one embodiment, the upper plunger 117 is disposed through the upper channel 163B defined by the frame 101. In operation, as the main shaft 115 is rotated, the eccentric sheave 102 produces an up and down axial motion that is imparted to the upper plunger 117 through the upper plunger eyebolt 122 and eccentric strap 190. As such, movement of the upper plunger 117 in an up and down axial motion along axial directions A and B is caused by rotation of the eccentric sheave 102 by the main shaft 115 is rotated, while movement of the lower plunger 118 in a similar up and down axial motion along axial directions A and B that alternates with the up and down motion of the upper plunger 117 is caused by rotation of the lifting cam 104 by the main shaft 115 as described above. The upper punch 140 is disposed within the upper plunger 117 and secured in place using an upper plunger nut 131.

In some embodiments, the lower plunger 118 is disposed through the lower channel 164B of frame 101. As shown, the lower plunger 118 is operatively coupled with a lower adjusting nut 111 which is rotated to adjust the height of the lower punch 141 relative to the lower plunger 118 and therefore control the size of the pellet (e.g., the length of the pellet). In addition, an upper adjusting nut 110 is provided to control the flushness of the lower punch 141 relative to the die 138. As shown, the combination of an adjusting nut collar 132, adjusting nut clip 133 and adjusting nut clip screw 134 engages the upper and lower adjusting nuts 110 and 111 to the lower plunger 118 for adjustment of the lower punch 141. A lower plunger bushing 119 is coupled to the bottom end of the lower plunger 118.

As shown, the main shaft 115 is also engaged to a swivel cam 105 that defines an eccentric pathway (not shown) configured to receive a shaker roller pin 128, wherein the shaker roller pin 128 is in operative engagement with a swivel lever roller arm 129 defined by the swivel lever 107. The swivel lever roller arm 129 imparts a back and forth or rocking motion to the swivel lever 107 as the swivel lever roller arm 129 travels along the eccentric pathway defined by the swivel cam 105 as the main shaft 115 rotates. In addition, the swivel lever 107 is configured to receive a spring 108 and a swivel lever fulcrum pin 130 which is attachable to the frame 101 and collectively facilitate the back and forth motion of the swivel lever 107 imparted by the swivel cam 105 as the main shaft 115 rotates. In some embodiments, a tensioner pin 106 may be provided that ensures the top of the spring 108 is maintained at the appropriate location relative to the swivel lever 107. In some embodiments, a collar 136 is disposed through the swivel cam 105 for engagement with the main shaft 115.

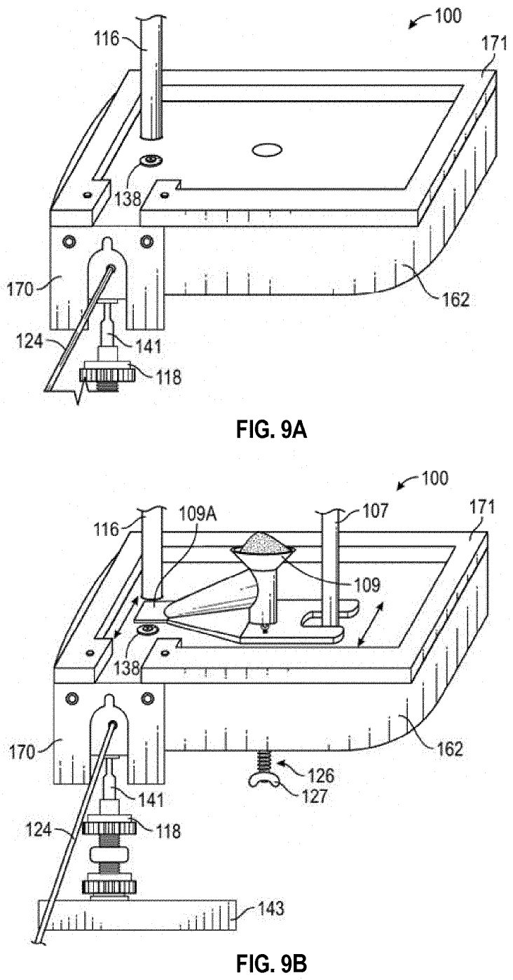

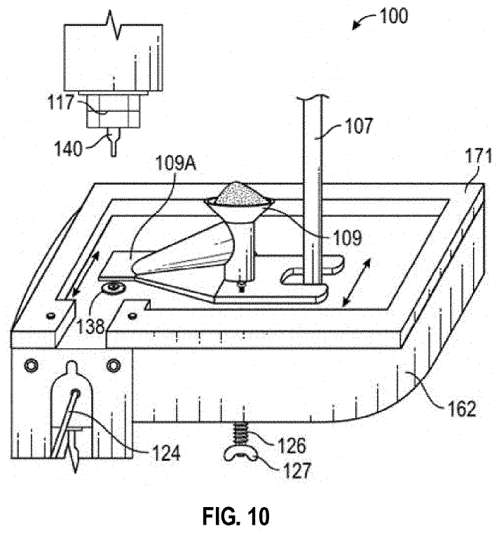

As shown in FIGS. 9A-9B and 10, the swivel lever 107 is in operative engagement with a feed cup 109 that is operable to deposit a predetermined amount of a pharmaceutical substance in powder form into the die 138 for formation of a pellet in the stamping operation. In some embodiments, the feed cup 109 may be in operative association with a hopper 151 (FIG. 4) that is configured to provide a storage conduit for supplying the pharmaceutical substance in powder form to the feed cup 109, although in other embodiments the feed cup 109 may be configured to store and dispense the pharmaceutical substance without a hopper 151. In some embodiments, the feed cup 109 may further include an elongated edge 109A positioned above the die 138 and below the upper punch 140. The feed cup 109 may also define a forked end 109B on the opposite side, wherein the forked end 109B is operable to capture the swivel lever 107, as shown in FIG. 9B. In one embodiment, the forked end 109B of the feed cup 109 couples the swivel lever 107 to the feed cup 109 such that the feed cup 109 is operable to swivel between a supply position wherein the hopper 151 supplies an amount of pharmaceutical substance into the feed cup 109 and a dispensing position wherein the feed cup 109 is aligned directly over the die 138 and dispenses an amount of pharmaceutical substance in powder form to the die 138 when shaken by the back and forth operation of the swivel lever 107 before the feed cup 109 swivels to the non-dispensing position, where the feed cup 109 is no longer positioned directly over the die 138. During this swiveling operation, the elongated edge 109A swivels across an upper side of the die 138, an operation which will facilitate the ejection of the formed pellet. This swiveling operation of the feed cup 109 between non-dispensing and dispensing positions is repeated for the formation of each individual pellet.

As noted above, the upper plunger 117 is engaged to the upper punch 140 to drive the upper punch 140 in an axial direction A and then axial direction B, while the lower plunger 118 is engaged to a lower punch 141 to drive the lower punch 141 in an opposite axial direction B and then axial direction A as illustrated in FIGS. 3-5. In this arrangement, the upper punch 140 and lower punch 141 are driven into contact with the die 138 in alternating sequence against the die 138 in an automated stamping operation as the upper plunger 117 and lower plunger 118 are actuated by operation of the motor 150 in alternating sequence relative to the die 138 as the main shaft 115 is rotated.

In some embodiments, as shown in FIG. 4 one or more oil cups 112 may be provided to supply a lubricant along the moving components of the automated pellet press 100. For example, a respective oil cup 112 may supply lubricant along the eccentric strap 103 as well as first and second channels 167 and 168 of respective first and second shoulders 165 and 166 of frame 101. In some embodiments, an oil cup 113 defining an elbow may provide a lubricant to the upper plunger eyebolt 120.

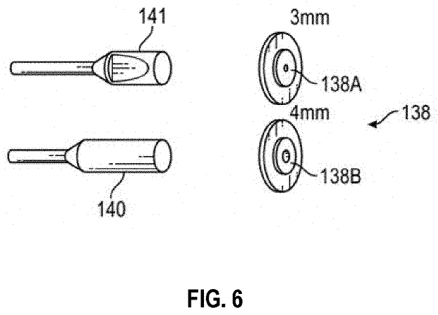

Referring to FIG. 6, embodiments of the upper and lower punches 140 and 141 and the die 138 are illustrated. In some embodiments, the upper punch 140 has a shorter length than the lower punch 141 and each may have a 3 mm or 4 mm width. Similarly, a die 138A may be configured to receive the 3 mm upper and lower punches 140/141 or a die 138B may be continued to receive the 4 mm upper and lower punches 140/141 in an alternate embodiment. In one aspect, the upper and lower punches 140/141 may have different sizes to comport with the size of the die 138 used to form the pellets of a particular shape and size during the stamping operation.

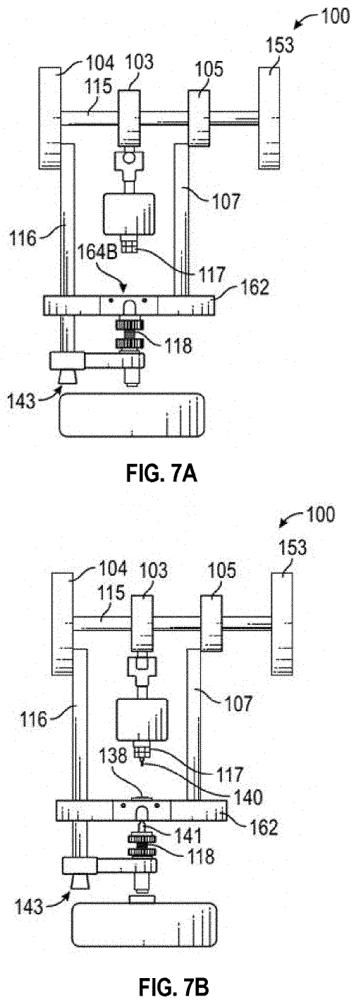

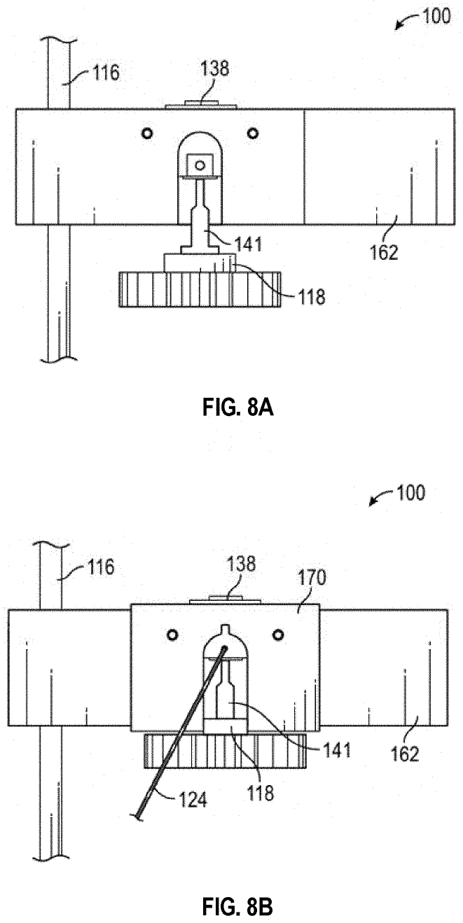

Referring to FIGS. 7A and 7B as noted above, the upper punch 140 is in operative engagement with the upper plunger 117 and the lower punch 141 is in operative engagement with the lower plunger 118. Upon assembly, the upper punch 140 is inserted into the upper plunger 117 and the lower punch 141 is inserted into the lower plunger 118. Upon assembly, the die 138 is inserted into the lower channel 164B. Referring to FIGS. 8A and 8B, in some embodiments a vacuum tubing 124 is in operative association with the die 138 such that suction is provided by the vacuum tubing 124 to facilitate the deposition of powder into the die 138. As shown in FIG. 8B, some embodiments of the lower mounting portion 162 of the frame 102 may have a face plate 170 affixed to the lower mounting portion 162 for protection. Referring to FIG. 9A, a top plate 171 is mounted onto the lower mounting portion 162 and secured to the face plate 170. Referring to FIG. 4, the feed cup 109 may be mounted to the top plate by the feed cup bolt 125, wherein the feed cup bolt 125 is sheathed by the feed cup spring 126 and secured by the feed cup nut 127. As noted above, the feed cup 109 is coupled to the swivel lever 107 by a forked end 109B of the feed cup 109 such that the feed cup 109 is operable to swivel between a dispensing position and a non-dispensing position.

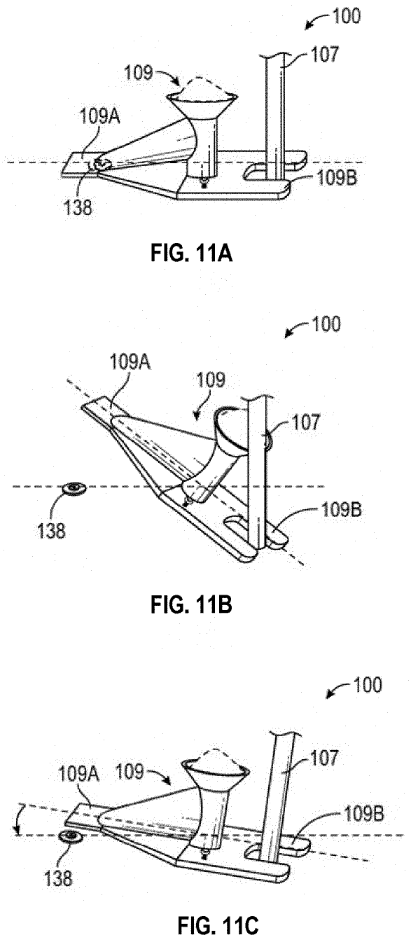

The dispensing position, as shown in FIG. 11A, involves swiveling the elongated end 109A of the feed cup 109 over the die 138 such that an amount of powdered material is dispensed into the die 138. The elongated end 109A of the feed cup 109 is then swiveled into a non-dispensing position away from the die, as shown in FIG. 11B. While the feed cup 109 is in the non-dispensing position, the upper and lower punches 140/141 contact the die 138 in alternating sequence and stamp the powdered material into a pellet. The feed cup 109 is then returned to the dispensing position, however, as shown in FIG. 9C, the lower punch 141 lifts the pellet out of the die 138 and the elongated edge 109A of the feed cup 109 contacts and expels the pellet out of the die 138 and into a repository (not shown), thus ejecting the pellet from the die 138 in time to fill the die 138 with more powdered material for forming another pellet.

One method of manufacturing pellets using the automated pellet press 100 as disclosed herein shall be discussed. As noted above, a predetermined amount of a powdered material, such as a pharmaceutical substance, is first deposited into the die 138 by feed cup 109. Once the powdered material is deposited into the die 138, the feed cup 109 swivels away from the dispensing position and the lower plunger 118 is actuated in axial direction B such that the lower punch 141 contacts the die 138 and sets the powdered material within the die 138. After the die 138 is contacted by the lower punch 141, the upper plunger 117 then drives the upper punch 140 into contact the die 138 from opposite axial direction A to fully form the pellet within the die 138 from the deposited powder material. The lower plunger 118 then subsequently drives the lower punch 141 into contact with the die 138 again from axial direction B to extract and remove the formed pellet from the die 138, lifting the formed pellet in an axial direction B out of the die. After the lower punch 141 lifts the formed pellet from the die 138, the feed cup 109 swivels back into the dispensing position again to dispense another amount of powdered substance into the die 138 for formation of another pellet by the upper and lower punches 140 and 141 in the stamping operation. During the swiveling operation of the feed cup 109 shown in FIG. 11C, the elongated edge 109A of the feed cup 109 concurrently knocks the formed pellet having been lifted by the lower punch 141 out of alignment with the die 138 for collection. As such, the three step stamping operation of the upper and lower punches 140 and 141 set, form, and extract each pellet from the die 138.

In some embodiments, as shown in FIG. 8B, vacuum tubing 124 is in communication with the die 138 to apply a vacuum or suction to the interior portion of the die 138 to facilitate the formation of the deposited powder material within the die 138 prior to the stamping operation between the upper and lower punches 140 and 141.

In some embodiments as shown in FIG. 4, the rotatable main shaft 115 defines a first key 146 for preventing slippage of the lifting cam 104 from the rotatable main shaft 115, a second key 145 for preventing slippage of the eccentric sheave 102 from the rotatable main shaft 115, and a third key 147 for preventing slippage of the swivel cam 105 from the rotatable main shaft 115.

### Automated Pellet Vialing and Labeling

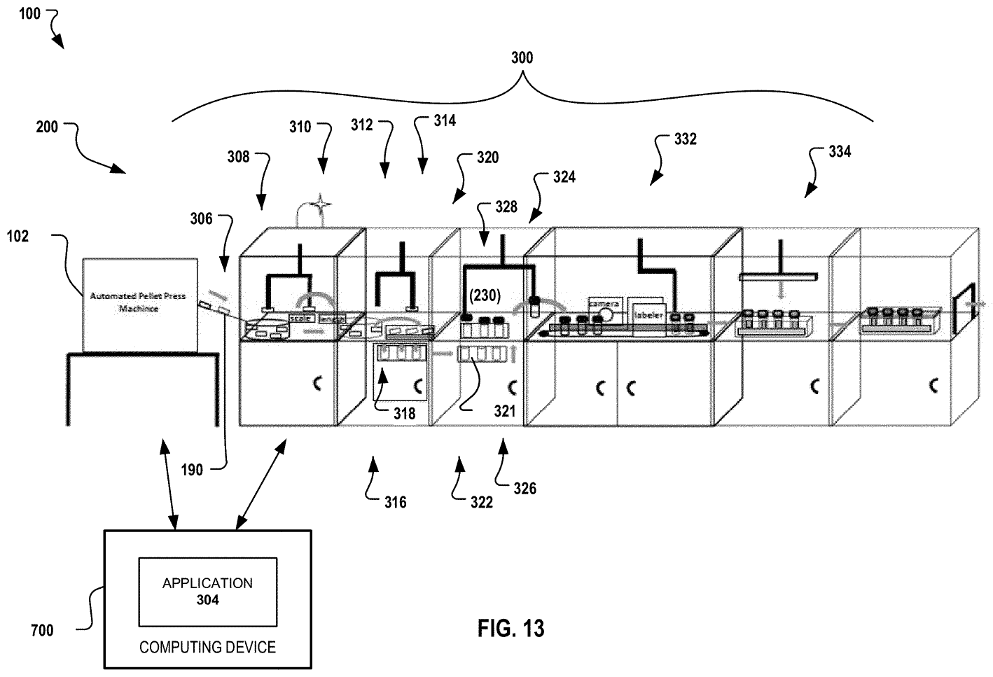

As shown in FIG. 13, the automated pellet press 100 and the pellet vialing apparatus 200 may be in operable communication with one another and collectively form a fabrication line 300 for pellet production and vialing, operable for the automation of transitioning the pellets 190 to a finished (vialed) product which may be labeled as described herein. In some embodiments, the fabrication line 300 may generally include at least a ramp 306 for transporting the pellets 190 from the automated pellet press 100, and a repository 308 where pellets 190 may be collected and stored prior to vialing. In some embodiments the fabrication line also includes a weight and length station 310 where pellets 190 may be checked for quality assurance for meeting one or more manufacturing standards and at least one of a routing arm 312 as well as a belt conveyance 314 for transporting the pellets 190 between modules. In the present disclosure, the fabrication line also includes a vialing module 316 where pellets 190 may be vialed into vials 106 containing the pellets 190. The vialing module 316 includes pre-vialing cavities 318 of a tray 321 where pellets 190 may be sorted, and a capping module 324 wherein vials 106 are capped, as depicted and further described herein.

In some embodiments, the fabrication line 300 may be in operable communication with a computing device 302, executing an application 304. The computing device 700 may include a server workstation with at least one server, a controller, a personal computer, a terminal, a workstation, a portable computer, a mobile device, a tablet, a mainframe, or other such computing device. The computing device 700 may be configured, by virtue of the application 304, to send and receive information and to send instructions to either of the automated pellet press 100 or the pellet vialing apparatus 200, via a network (which may include the Internet, an intranet, a virtual private network (VPN), and the like. In some embodiments, a cloud (not shown) may be implemented to execute one or more components of the computing device 302. In addition, aspects of either of the computing device 700 or the application 304 may be provided using platform as a service (PaaS), and/or software as a service (SaaS) using e.g., Amazon Web Services, or other distributed systems.

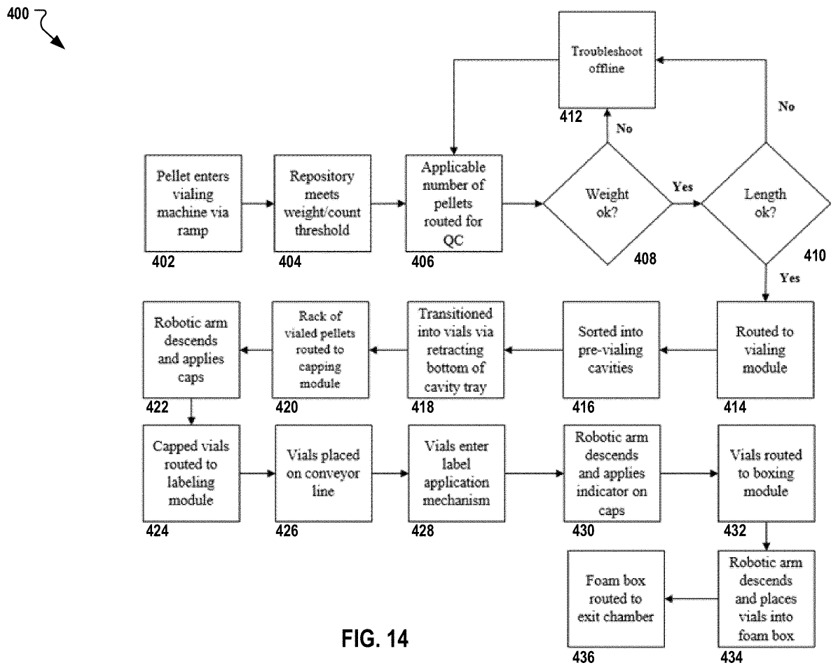

Referring to FIG. 14, a process flow 400 is depicted for automated vialing and labeling of the pellets 190. The pellets 190 are initially formed using the automated pellet press 100. In block 402, each of the pellets 190 enters the repository 308 of the pellet vialing apparatus 104 via the ramp 306. The ramp 306 may define a generally planar surface such that the pellets 190 slide freely along the ramp 306 from the automated pellet press 100 to the pellet vialing apparatus 200 (as a change in orientation of the ramp 306 may increase the transfer speed). The ramp 306 may be angled from the automated pellet press 100 to the pellet vialing apparatus 100 in any predetermined manner suitable for transferring the pellets 190 as described. In some embodiments, the ramp 306 defines an angled stainless steel ramp.

As indicated in block 404, the repository 308 may define a weight and/or count threshold, and it is determined whether this threshold has been met. The threshold may be predetermined by pellet strength and a required sample size intra-batch per quantity produced. In some embodiments, aspects of this production quality assurance may be measured and monitored by the application 304.

As indicated in block 406, an applicable number of the pellets 190 may then be transported to the weight and length station 310 via the routing arm 312 and the belt conveyance 314 for quality control. As indicated in decision blocks 408 and 410, weight and length of the pellets 190 is measured at the weight and length station 310, which may include an embedded scale for weight measurement, and an embedded micrometer device for length measurement. In some embodiments, a signal light (which may be yellow or other colors) may be included with the fabrication line 300 and may be illuminated to indicate that the intra-batch segment is on hold until acceptable measurements are achieved. As indicated in block 412, if an intra-batch segment of the pellets 190 does not meet certain predefined measurements (weight and length), the process may pause and/or a technician troubleshoot and manually review offline.

Referring to blocks 414 and 416, pellets 190 that satisfy the weight and length measurement thresholds are routed to the vialing module 316 and sorted into the pre-vialing cavities 318. Signal lights may be implemented to indicate that the pellets 190 have satisfied the measurement thresholds. The pre-vialing cavities 318 of the fabrication line 400 may be substantially equal to or equivalent to the size of the individual pellets 190, and be angled at a predetermined decline to accommodate transition of the pellets 190 into respective vials 320. In some embodiments, the pre-vialing cavities 318 are defined within the tray 321 and positioned over a sliding solid base 322.

Referring to block 418, the pellets 190 positioned within the pre-vialing cavities 318 may be transitioned into respective vials 320. Below the sliding base 322, vials 320 are loaded directly under the pre-vialing cavities 318 and may be in direct alignment with the pre-vialing cavities 318 above accounting for the angled decline. The tray 321 may then be triggered to retract, which may be initiated upon sensors verifying the presence of mass in the pre-vialing cavities 318, such that the pellets 190 slide into the vials 320, thereby fabricating vialed pellets 190, as indicated in FIG. 2. The tray 321 then returns into an original position.

Referring to blocks 420 and 422, the vials 106 containing pellets 190 may then be routed to the capping module 324. In this manner, the vials 106 containing pellets 190, which may remain in the tray 321 to accommodate alignment, can then be migrated to the capping platform 326 which is aligned to the capping grid 328 above and transitions down and applies a cap 330 to each of the vials 106 containing pellets 190, with a twisting motion.

Referring to block 424, the vials 106 containing pellets 190 with caps 330 applied to the vials 106 are then routed to the labeling module 332. Referring to blocks 426, 428, and 430, the vials 106 are removed from the tray 321 and placed into a linear feed of the labeling module 332 that consists of a conveyor belt and fitted side walls. The labeling module 332 introduces vials 106 one by one into a labeling mechanism that applies labels (not shown) with a perforated line directly in between the cap 330 and the top end of the vial container. In some embodiments, prior to total passage through the labeling module 332, pre-labeled images may be taken using a camera (not shown). In some embodiments, once the labels are applied to the vials 106 a robotic arm of the labeling module 332 applies an e-beam indicator.

Referring to block 432, the vials 106 with labels applied or otherwise having been transitioned through the labeling module 332 are transferred to a boxing module 340. Referring to blocks 436 and 434, a robotic arm of the boxing module 340 is implemented to descend and move vials 106 containing pellets 190 into a box or other container. In some embodiments, the box may be a foam insert box in accordance with e-beam dose map validation configuration. The box is then loaded into an exit chamber and made available to quality assurance prior to storage in quarantine awaiting sterilization results.

In some embodiments, the finished vials 106 may have the pellets 190 enclosed within a small glass container, which may be cylindrical defining a screw threading portion for engaging with the caps 330. The vials 106 may comprise glass or plastic and may define an amber color for protecting the pellets 190 against ambient light or other environmental contaminants.

In other embodiments, the vials 106 may further include an insert positioned within each vial 106 proximate to or in direct contact with the pellets 190. The insert may be comprised of glass or plastic similar to the vials 320 and may be useful for maintaining the pellets 190 within a fixed position relative to the vials 106.

Referring to FIG. 2, a process flow 500 related to the aforementioned pellet vialing framework is indicated. In block 502, a fabrication line 300 is provided including the automated pellet press 100 and the pellet vialing apparatus 200. In block 504, a plurality of pellets 190 are formed using the automated pellet press 100, and in block 506, the plurality of pellets 190 are transitioned to the pellet vialing apparatus 200. In block 508, it is determined whether the plurality of pellets 190 satisfies predetermined measurement thresholds associated with weight and length related to manufacturing standards. In block 510, each of the plurality of pellets 190 is sorted into respective cavities of a tray. In block 512, the tray is repositioned, or tipped to pass the plurality of pellets 190 to within a plurality of respective vials 106. In block 514, the vials 106 are capped and may then be labeled, boxed, and stored.

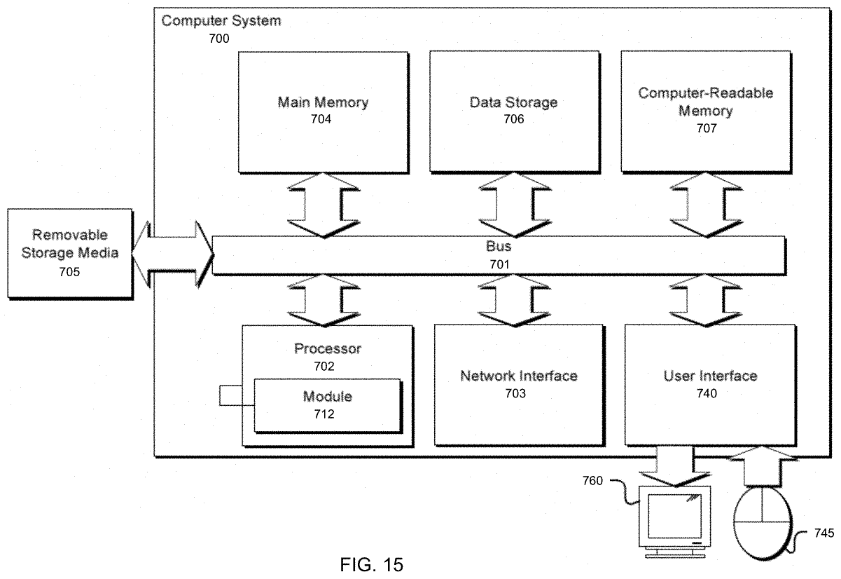

FIG. 15 is an example schematic diagram of a computing device 700 that may implement various methodologies discussed herein. For example, the computing device 700 may comprise the computing device 302 executing aspects of the application 304. The computing device 700 includes a bus 701 (i.e., interconnect), at least one processor 702 or other computing element, at least one communication port 703, a main memory 704, a removable storage media 705, a read-only memory 706, and a mass storage device 707. Processor(s) 702 can be any known processor, such as, but not limited to, an Intel® Itanium® or Itanium 2® processor(s), AMD® Opteron® or Athlon MP® processor(s), or Motorola® lines of processors. Communication port 703 can be any of an RS-232 port for use with a modem based dial-up connection, a 10/100 Ethernet port, a Gigabit port using copper or fiber, or a USB port. Communication port(s) 703 may be chosen depending on a network such as a Local Area Network (LAN), a Wide Area Network (WAN), or any network to which the computer device 700 connects. Computing device may further include a transport and/or transit network 755, a display screen 760, an I/O port 740, and an input device 745 such as a mouse or keyboard.

Main memory 704 can be Random Access Memory (RAM) or any other dynamic storage device(s) commonly known in the art. Read-only memory 706 can be any static storage device(s) such as Programmable Read-Only Memory (PROM) chips for storing static information such as instructions for processor 702. Mass storage device 707 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices, may be used.

Bus 701 communicatively couples processor(s) 702 with the other memory, storage, and communications blocks. Bus 701 can be a PCI/PCI-X, SCSI, or Universal Serial Bus (USB) based system bus (or other) depending on the storage devices used. Removable storage media 705 can be any kind of external hard drives, thumb drives, Compact Disc-Read Only Memory (CD-ROM), Compact Disc-Re-Writable (CD-RW), Digital Video Disk-Read Only Memory (DVD-ROM), etc.

Embodiments herein may be provided as a computer program product, which may include an apparatus-readable medium having stored thereon instructions which may be used to program a computer (or other electronic devices) to perform a process. The apparatus-readable medium may include, but is not limited to optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/apparatus-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

As shown, main memory 704 may be encoded with the application 304 that supports functionality discussed above. In other words, aspects of the application 304 (and/or other resources as described herein) can be embodied as software code such as data and/or logic instructions (e.g., code stored in the memory or on another computer readable medium such as a disk) that supports processing functionality according to different embodiments described herein. During operation of one embodiment, processor(s) 702 accesses main memory 704 via the use of bus 701 in order to launch, run, execute, interpret, or otherwise perform processes, such as through logic instructions, executing on the processor 702 and based on the application 304 stored in main memory or otherwise tangibly stored.

The description above includes example systems, methods, techniques, instruction sequences, and/or computer program products that embody techniques of the present disclosure. However, it is understood that the described disclosure may be practiced without these specific details. In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include an apparatus-readable medium having stored thereon instructions, which may be used to program a computer system (or other electronic devices) to perform a process according to the present disclosure. A apparatus-readable medium includes any mechanism for storing information in a form (e.g., software, processing application) readable by an apparatus (e.g., a computer). The apparatus-readable medium may include, but is not limited to optical storage medium (e.g., CD-ROM); magneto-optical storage medium, read only memory (ROM); random access memory (RAM); erasable programmable memory (e.g., EPROM and EEPROM); flash memory; or other types of medium suitable for storing electronic instructions.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” or “module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

It is believed that the present disclosure and many of its attendant advantages should be understood by the foregoing description, and it should be apparent that various changes may be made in the form, construction, and arrangement of the components without departing from the disclosed subject matter or without sacrificing all of its material advantages. The form described is merely explanatory, and it is the intention of the following claims to encompass and include such changes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method for manufacturing pellets, comprising:
providing an automated pellet press, wherein the automated pellet press comprises:
a fixed die secured to a frame, the die configured to receive a powder material;
a lower punch positioned axially on one side of the fixed die; and
an upper punch positioned axially on an opposite side of the fixed die and opposite to the lower punch;
wherein the opposing upper and lower punches are operable for pressing the powder material into a formed pellet within the die;
setting a powder material into the fixed die using the automated pellet press, wherein the lower punch or upper punch is operable to contact the powder material for setting the powder material into the fixed die;
pressing the set powder material into a formed pellet using the automated pellet press, wherein the upper punch or lower punch is operable to contact the powder material for pressing the powder material into the fixed die to form a pellet;
ejecting the pellet from the fixed die;
collecting the pellet into a repository of a vialing apparatus;
sorting one or more pellets into cavities of a tray of a vialing apparatus; and
depositing the one or more pellets into one or more respective vials.

2. The method of claim 1, wherein the fixed die defines a cavity configured to shape the powder material into a pellet.

3. The method of claim 1, wherein the lower punch is driven in an axial direction by a lower plunger operatively secured to the lower punch, and wherein the upper punch is driven in an axial direction opposite to the axial direction of the lower punch by an upper plunger operatively secured to the upper punch.

4. The method of claim 1, wherein the powder material comprises a pharmaceutical powder.

5. The method of claim 1, wherein the pellet is ejected from the automated pellet press by the lower punch or upper punch which contacts and ejects the pellet from the fixed die.

6. The method of claim 1, wherein the one or more pellets are transported to the repository of the vialing apparatus using an angled ramp.

7. The method of claim 1, wherein the one or more pellets are transported between modules of the vialing apparatus using a belt conveyance.

8. A method for manufacturing vials of pellets, comprising:
setting a powdered material onto a fixed die using an automated pellet press, wherein the fixed die is contacted by a lower punch or an upper punch in an axial direction to set the powdered material;
pressing the powdered material onto the fixed die using an automated pellet press, wherein the fixed die is contacted by the lower punch or the upper punch in an opposite axial direction to press the powdered material into a plurality of formed pellets;
transporting the plurality of formed pellets to a repository of a pellet vialing apparatus, wherein the plurality of formed pellets are transported along an angled surface from the automated pellet press to the pellet vialing apparatus;
sorting the plurality of formed pellets into cavities of a tray, wherein each cavity of the tray is situated in direct alignment with one of a plurality of vials; and
depositing the plurality of formed pellets from the cavities of the tray into one of the plurality of vials by retracting the tray such that one or more of the plurality of pellets slide into the one of the plurality of vials.

9. The method of claim 8, further comprising:
determining whether the plurality of formed pellets meets predetermined manufacturing standards, wherein the plurality of formed pellets are transported to a station operable for determining whether the plurality of formed pellets are within suitable weight and length parameters based on the predetermined manufacturing standards.

10. The method of claim 9, wherein one or more of the plurality of formed pellets that does meet predetermined manufacturing standards is transported to a sorting module.

11. The method of claim 9, further comprising:
triggering an alert when one of more of the plurality of pellets does not meet the predetermined manufacturing standards.

12. The method of claim 11, further comprising:
pausing the method of manufacturing when an alert is triggered.

13. The method of claim 8, wherein the tray is moved through the fabrication line using a sliding base.

14. The method of claim 8 wherein the retracting motion is triggered by one or more sensors.

15. The method of claim 13, wherein a retracting motion of the retracting tray has enough acceleration to overcome static friction between the plurality of formed pellets and the tray, in which the plurality of pellets is in contact.

16. The method of claim 8, further comprising:
capping the plurality of vials;
labeling the plurality of vials; and
boxing the plurality of vials.

17. A system for manufacturing pellets, comprising:
an automated pellet press, wherein the automated pellet press comprises:
a fixed die secured to a frame;
a lower punch positioned axially on one side of the fixed die wherein the lower punch is driven by a lower plunger in an axial direction; and
an upper punch positioned axially on an opposite side of the fixed die and opposite to the lower punch, wherein the upper punch is driven by an upper plunger in an opposite axial direction;
wherein the opposing upper and lower punches are operable for pressing a powder material into a formed pellet within the fixed die in a stamping motion by the upper and lower punches; and
a vialing apparatus, wherein the vialing apparatus comprises a processor in operative communication with a sequence of modules, wherein the sequence of modules are operable for sorting and depositing the plurality of pellets into a respective one of a plurality of vials.

18. The system of claim 17, wherein a powder material disposed on the fixed die is contacted by the upper punch or the lower punch in an axial direction to set the powder material into the fixed die.

19. The system of claim 17, wherein a powder material disposed on the fixed die is contacted by the upper punch or lower punch in an opposite axial direction to form the powder material into a pellet.

20. The system of claim 17, wherein the lower punch or the upper punch is operable for ejecting the formed pellet from the fixed die.
